(19) United States
(12) Patent Application Publication
(10) Pub. No.: US 2025/0344957 A1
(43) Pub. Date: Nov. 13, 2025

(54) DEVICE FOR BLOOD PRESSURE MEASUREMENT

(71) Applicant: iNDTact GmBH, Wurzburg (DE)

(72) Inventors: Raino PETRICEVIC, Wurzburg (DE); Clemens LAUNER, Wurzburg (DE)

(21) Appl. No.: 18/870,441

(22) PCT Filed: May 31, 2023
(86) PCT No.: PCT/EP2023/064528

§ 371 Date: Nov. 28, 2024

(30) Foreign Application Priority Data
Jun. 1, 2022 (DE) 10 2022 113 794.5

(51) Int. Cl. A61B 5/021; A61B 5/00

(52) U.S. Cl. CPC A61B 5/02141; A61B 5/6826; A61B 2562/04

(57) ABSTRACT

A device (1, 12, 17, 22, 26, 32, 37, 42) for blood pressure measurement, comprising a support (2), a bending sensor (4) arranged on the support (2) and designed to detect when the support (2) is bent, two legs (3) that are arranged at an angle to the support (2) at opposite sides of the support (2), and an evaluation unit (9) designed to determine a blood pressure value on the basis of sensor signals emitted by the bending sensor (4). The invention also relates to a method for measuring blood pressure.

### TECHNICAL FIELD

The invention relates to a device for blood pressure measurement.

### BACKGROUND

A blood pressure monitor, also known as a sphygmomanometer, is a measuring device that can be used to measure a patient's arterial pressure externally, on the upper arm or wrist. The measuring devices, which work according to different methods, display the upper (systolic) and lower (diastolic) arterial pressure with varying degrees of accuracy.

The commercially available measuring devices measure the arterial pressure on the inside of the wrist and are easy to use. The measuring device and cuff form a single unit. The device is placed on the inside of the wrist, on the pulse artery, and attached to the cuff. After starting the measurement, the cuff is inflated by an electric pump to an initial measurement pressure until no more blood can flow through the artery. The pressure in the cuff is gradually reduced by an electrically controlled valve. Sensors record the current pressure and the changing blood flow sounds. The device uses pattern recognition to register the systolic and diastolic arterial blood pressure points. In addition, other key figures such as pulse rate and cardiac arrhythmia can be recorded and an overall assessment status determined.

Modern upper arm measuring devices display the values on an integrated screen. The cuff can be separated from the measuring device to allow for different cuff sizes. They are less user-friendly than measuring devices that measure on the inside of the wrist and are more expensive. However, the accuracy of the measurement results speaks in their favor.

In indirect arterial pressure measurement, often abbreviated as RR (according to Riva-Rocci) or more rarely as “NIBP” (“non-invasive blood pressure”), the arterial pressure is measured on an extremity, usually the arm, using a blood pressure monitor.

During the auscultatory measurement, a pressure cuff of a suitable width is inflated on the upper arm to above the expected arterial pressure. During slow deflation, the appearance and then disappearance of a Korotkow sound can be heard (auscultation) over the artery in the arm using a stethoscope. The pressure that can be read on the scale of the measuring device when the sound is first heard corresponds to the upper systolic arterial pressure value, i.e. the systolic pressure at this moment is greater than the pressure of the cuff. The pressure is released further at a suitable rate. If the cuff pressure falls below the minimum arterial pressure value, the noise disappears. This value is referred to as the diastolic pressure and is noted as the lower value. The auscultatory measurement is the standard non-invasive measurement method.

A pressure cuff is also applied to the upper arm during the palpatory measurement. When the pressure is released, the pulse is palpated on the radial artery. The pressure that can be read on the scale of the measuring device when the pulse is palpated for the first time corresponds approximately to the upper systolic arterial pressure value. The diastolic value cannot be determined in this way. This method is suitable for noisy environments, for example, especially in the emergency services. Even less accurate is the palpatory measurement without a pressure cuff, in which the radial artery is palpated with two fingers and the blood vessel is compressed with the finger closest to the heart until no pulse can be felt with the finger furthest from the heart. The force exerted by the finger closest to the heart is an approximate indication of the blood pressure.

In principle, the oscillatory measurement is carried out in the same way as the other two methods; the upper and lower values are estimated using the amplitude curve of a pulse-synchronized pointer deflection on the measuring device, which represents the transmission of vibrations from the vessel wall to the pressure cuff. With manual measurement, only imprecise results can be obtained with this method. However, this measuring method is used quite reliably by automatic measuring devices for continuous monitoring, e.g. post-operatively in the recovery room. As an alternative to continuous invasive pressure measurement, they measure the patient's arterial pressure at intervals of a few minutes. The oscillatory measuring method is also used in the wrist measuring devices that are now in widespread use.

Long-term blood pressure measurement (ABDM) is based on the same principle. The patient wears a blood pressure cuff permanently (usually for a whole day), which inflates and measures automatically at set intervals, as well as a recording device. This method is considered the gold standard for detecting and assessing the severity of arterial hypertension.

A measurement using pulse wave analysis is also possible. This involves interpreting optical signals such as the pulse formation of the arteries and estimating the blood pressure from this data. The advantage of this method is that the measurement is permanent and non-invasive and only requires a wristband without a pressure cuff.

The following publications are examples of the relevant prior art and relate to devices for measuring blood pressure, their components and associated measuring methods: DE 3004011 A1, DE 3632592C2, DE 4439253 A1, DE 10214220 A1, EP 0165505 A1, EP 0334652 B1, EP 0467 853 A1, WO 2005/046466 A1, WO 2009/141171 A2, EP 0744155 A1, US 5 025 793, US 2012/0238887A1, US 2013/0226015 A1, US 2019/0320980 A1, US2019/0374116 A1, WO2017/183106 A1, WO 2018/231711 A1, WO 2019/209679 A1, WO 2020/112555 A1, WO 2021/110597 A1.

The disadvantage of these conventional devices and methods is that inflating the cuff can be very uncomfortable or even painful. This can cause skin lesions, especially in older people. These measurement methods are particularly unpleasant in the case of thin skin, inflammation and repeated measurements at high frequency and can cause pressure sores over large areas.

Although WO 2009/141171 A2 proposes a pressureless blood pressure measurement, the sensor disclosed there requires an additional cuff to be pressurized with air pressure for calibration. This calibration usually has to be repeated each time the cuff is put on. This type of blood pressure measurement is therefore cumbersome.

There are therefore no simple and, above all, compact systems that manage entirely without such unpleasant pressurization by means of air cushions.

The invention is therefore based on the task of providing a device for blood pressure measurement which is simple in design and does not have an air cushion for pressurization.

### SUMMARY OF INVENTION

To solve this problem, a device for blood pressure measurement with the features of claim 1 is provided.

The device for blood pressure measurement according to the invention comprises a support, a bending sensor arranged on the support, which is designed to detect a bend in the support, two legs arranged on opposite sides of the support at an angle to the support, and an evaluation unit designed to determine a blood pressure value using sensor signals from the bending sensor.

The invention is based on the idea that the bending sensor is bent by the arterial and venous pulsation of the blood pressure, so that the sensor signals generated can be evaluated in order to determine the blood pressure. The bending exerted on the bending sensor generates a tensile stress, which causes the sensor signals. The device for blood pressure measurement according to the invention enables a particularly sensitive measurement of the blood pressure or the temporal course of the blood pressure, whereby only a low pre-tensioning force is required compared to the measuring devices known in the prior art. This avoids an unpleasant feeling of pressure and discomfort for the patient. The device for blood pressure measurement according to the invention is therefore also particularly suitable for long-term monitoring of a patient.

In the context of the invention, it is preferred that the bending sensor is a piezoelectric sensor. Such sensors are small and are characterized by a high sensitivity.

Preferably, the bending sensor can be designed as a bimorph sensor arrangement with two individual sensors arranged around the neutral fiber. The individual sensors have anti-parallel polarity and are arranged symmetrically around the neutral fiber. When the bimorph sensor arrangement arranged on the support is bent in one direction, one of the individual sensors is stretched, while the other individual sensor is equally compressed. When the two individual sensors are subjected to such an opposing load, their signals add up.

It is also within the scope of the invention that the bending sensor of the device according to the invention is designed as a multimorph bending sensor and has several pairs of individual sensors with alternating antiparallel polarity. The sensitivity can be increased by providing several pairs of individual sensors.

A further development of the invention provides for one or both legs to be hinged to the support. The articulated attachment enables adaptation to body parts of different sizes, for example to fingers of different sizes. One or both legs of the support can have a support. Preferably, the support is designed in such a way that there is a defined connection to an underlying vessel such as an artery or vein. Preferably, the support can have a narrow, linear elevation that can be placed vertically on the vessel. In the case of a device that can be worn on the finger, the support is preferably in the shape of a circular segment.

The device for blood pressure measurement according to the invention can have a clamping element in order to apply a defined force to one limb. Of course, both legs can also have such a clamping element. The device according to the invention thus comprises the bending sensor arranged on the support, the two angled legs, the evaluation unit and optionally at least one clamping element.

Pulse pressure-induced pulsation of a volume, in particular of a part of a patient's body on which a blood pressure measurement is to be taken, can be converted into a bend by a U-shape of the support. The clamping element is used to clamp the pulsating area of the volume between the legs of the U-shaped support with a preferably defined pre-tensioning force. In this way, the pulsation of the body part or a section of the body part, in particular a finger, caused by the pulse pressure can be converted into a bend by the U-shape of the support. This bend is detected by the bending sensor. The bending sensor is preferably located at the point where the bending is at its maximum.

A preferably constant contact pressure is applied to the body part via the clamping element. The clamping surface of the clamping element preferably covers the areas under which the pulsating vessels run. The clamping surface (=contact surface) of the clamping element is preferably designed in such a way that the force can be distributed evenly over the clamping surface. This can be achieved, for example, by a skin-friendly pad made of a flexible material such as rubber or foam or by means of an air cushion.

With such a device for measuring blood pressure according to the invention, a weak deflection caused by an arterial and/or venous pulsation of a body part at least partially clamped therein or surrounded thereby or its surface can be converted into a bending at the location of the bending sensor and thus into an electrical signal.

In particular, with the aid of such an arrangement, the arterial and/or venous pulsation in the finger caused by the blood vessels near the surface, which manifests itself as a pulsating local deflection of the surface or volume of certain points on the finger, can be mechanically transmitted to the bending sensor and converted into an electrical signal by the bending sensor, whereby the pulse pressure curve can be recorded by an electronic measuring system in the evaluation unit.

The recorded electrical sensor signal has a high signal-to-noise ratio. Due to the high signal quality of the electrical sensor signal, the pulse pressure curve can be reproduced in detail with all systolic and diastolic components.

Due to the high sensitivity of the device according to the invention, it is easily possible to detect the pulse signals with high accuracy even when using a comfortable pad and a 30 very low and therefore maximum comfortable fastening force. Thus, a deflection of one or both legs caused by a pulsation of the blood pressure triggers a bending of the wearer detected by the bending sensor, which can be evaluated by the evaluation unit. Preferably, the evaluation unit can determine the blood pressure over time.

Vital data (e.g. blood pressure values, pulse) can be determined by analyzing the characteristics (e.g. extreme values and their time intervals) of the pulse pressure curve.

Vital data can also be determined using artificial intelligence or machine learning, whose networks have previously been trained with a comprehensive patient database. In addition to the measurement curves, the training database can also contain any other medical data or patient diagnoses.

In the simplest case, the force exerted by the clamping element is constant. However, embodiments are also possible in which the force exerted by the clamping element is adjustable. In the device according to the invention, a clamping element can be supported on the support on the one hand and on a leg on the other.

As already mentioned, it is preferred that the support and/or the support comprising the legs of the device according to the invention is U-shaped.

The main components of the device for measuring blood pressure are described below. With regard to the sensor element, the preferred variant provides for at least one bending sensor, preferably a bimorph bending sensor arrangement with antiparallel polarity. Alternatively, however, multimorph bending sensors can also be used, which are composed of several sensor pairs (in the form of foils or bars) with alternating antiparallel polarity. The bimorph bending sensor arrangement is particularly preferably a piezoelectric bimorph bending sensor arrangement in which the sensors are piezoelectric sensors. Such a sensor element reacts very sensitively to bending. In principle, a bimorph bending sensor arrangement consists of two sensor layers that are arranged symmetrically around the neutral fiber (beam theory). When this arrangement is bent in one direction, one of the sensorically active bending sensor layers is stretched, while the other is equally compressed. When bent in the other direction, the opposite happens. Due to the antiparallel polarity of the two sensor layers, the signals of these opposing loads add up constructively (as they have the same sign) and increase the overall signal. Concurrent effects on the other hand (e.g. interfering temperature effects, pyroelectric effects) are largely canceled out and thus compensated. In addition to a bending sensor, the sensor element can also contain other sensor elements such as an optical sensor.

The support, on which the bending sensor is arranged, has the two legs. The support is therefore U-shaped and has at least one bending sensor, preferably a bending sensor element. If the legs of the U-shaped support are deflected against each other, this deflection results in a bend at the location of the sensor element.

By clamping a pulsating object volume, for example a finger, between the legs, the legs move against each other perpendicular to the clamped surface. With the appropriate design, even the smallest deflections can be transferred into a bend at the location of the sensor by the lever formed by the legs in relation to the support, so that these are detected by the bending sensor. The U-shaped support can also be designed as a ring that can be worn by a patient on the finger or arm.

In the device for measuring blood pressure according to the invention, the bending sensor is connected via a flexible or elastic unit in such a way that the device can be applied and clamped to the pulsating body part, for example a finger, with a defined fastening force. The pulsating vessels of the body part are located under a support of the limb or a support of the clamping element of the limb or touch the support of the clamping element.

Flexibility can be achieved by the clamping element having an elastic element, preferably a spring element. Preferably, the clamping element can be opened by applying force, for example via a lever mechanism. Particularly preferably, at least one joint with a resilient return mechanism can be provided to increase the flexibility of the clamping element. The resilient return mechanism can also comprise an electromechanical, pneumatic, hydraulic or piezoelectric actuator. In the case of a pneumatic actuator, for example, a manual pump, a multi-port valve and a pressure gauge can be provided to realize a defined, preferably constant force of the flexible clamping element. Instead of a manual pump, an electric pump with a control circuit can also be provided.

Preferably, the spring force of such a resilient reset mechanism can be adjusted manually or automatically. This can be done manually using a torque wrench, for example. It is particularly preferred that the spring force of such a resilient return mechanism is independent or at least largely independent of the deflection, at least within a certain tolerance range. Accordingly, the force of the clamping element is largely constant.

Flexibility can be realized by a spring or an elastic material that connects one or both clamping legs to the support. The support or supports of the leg or legs are designed in such a way that they can reproducibly clamp the part of the body at which the blood pressure is to be measured. For this purpose, the point on the patient's body part where the deflection is at its maximum is selected if possible. The support can be placed over the artery or vein so that it crosses the artery or vein. The support forms a defined force application point. The deflection of the support due to the pulsation of the artery or vein is transferred to the bending sensor and can be recorded by it.

The support can preferably adapt to the surface of the pulsating body part. For this purpose, the support of the clamping element can be at least partially padded. Preferably, the support or padding is made of skin-friendly material. In a particularly advantageous embodiment, the flexible part of the device for measuring blood pressure is shaped like a ring, e.g. a finger ring or wristband.

The force with which the clamping element is applied to the respective body part can be adjustable. This can be achieved, for example, by means of at least one adjustable spring. In a further advantageous embodiment, the device also contains an indicator for the clamping force. Such an indicator can be realized, for example, with a tension spring that transforms a small force into a large deflection of a force indicator with a mechanical transmission (e.g. lever, pulley or gear wheel).

The device can have an adjustability of the clamping force to a few fractions of a Newton. The adjustability can be in steps of 1/10 Newton, for example. However, it can also be in steps of up to 1/100 Newton. This allows the device for measuring blood pressure to be attached with a very small and at the same time defined clamping force. This is advantageous for determining absolute blood pressure values.

In another advantageous embodiment, a preset tension force generated by a spring regulates itself automatically when applied to the finger or another part of the body. This can be done electronically, for example, using a load cell in combination with an actuator.

In a particularly preferred embodiment, a compression spring that is as long as possible and as strongly pretensioned as possible is used, whose change in force in a limited adjustment range is only a fraction of the adjustment travel in relation to the total spring length.

Other versions of the device for measuring blood pressure according to the invention are also possible. The device can also be designed in a ring shape by miniaturization. The ring is preferably worn over the fingertip in front of the first joint. However, it is also possible to wear the ring anywhere on the finger. Such a ring can also be worn on the arm or leg or even on the neck, as the clamping force required for pulse pressure sensitivity is very low. The ring can therefore be designed as ring jewelry or arm jewelry or neck jewelry. Similarly, the ring can also be designed as part of a watch or smartwatch.

In a particularly advantageous embodiment, the device for measuring blood pressure comprises an at least partially elastic element, preferably a partially linear elastic element, e.g. a spiral spring, which at least partially surrounds the finger. In the following, this part is referred to as a ring element. By setting a defined clamping force, it is only necessary to calibrate the device according to the invention once. This means that the device can be delivered in a calibrated state.

By using several devices on different parts of the body, it is possible to make statements about certain vascular diseases in these parts of the body, for example. In addition, with at least two such rings at different positions (e.g. on the arm or finger), statements about the pulse wave velocity can easily be determined from the phase shift between the signals.

The evaluation unit comprises measurement electronics that contain an amplifier, signal conditioning electronics, an A/D converter and a radio and/or cable interface. It can also contain a display and various control elements, e.g. selection buttons. Ideally, the signal conditioning, the A/D converter and the radio transmission module of the measuring electronics are located on or in the support. This transmits the digitized signal to a monitoring gateway or a mobile display device, e.g. via Bluetooth or another wireless transmission method. This can also be a smartphone or a smartwatch. The support can also be designed as part of a smartwatch, which has all the components required to record and process the sensor signals, in particular measuring and electronic components assigned to the bending sensor. These components can also be in the form of miniaturized and integrated circuits such as FPGAs. Such circuits can be manufactured cost-effectively and easily integrated into a smartwatch or similar system.

However, the measuring electronics can also be at least partially connected to the bending sensor via a cable and placed in a housing at a different position on the patient's body, e.g. on the arm joint. This housing can also contain a display.

Surprisingly, the device according to the invention has shown that the pulse pressure curve can be recorded with an extraordinarily high signal quality. This means that even the smallest fluctuations in the pulse pressure curve, which could previously only be measured invasively, can be detected non-invasively. With the device according to the invention, more correlations and thus also clinical pictures can be revealed than was possible with previous non-invasive methods.

The course is evaluated using analytical evaluation of the curve characteristics such as peaks, e.g. maxima, minima, notch, peak shapes, relative peak positions, relative peak amplitudes, pulse frequency, areas under the peaks, areas under systolic and diastolic curve sections, and links between the characteristics.

The device according to the invention makes it possible to record vital data, in particular pulse and pulse pressure curve, at almost any point on the body, even through a soft pad, by attaching it to the corresponding part of the body in such a way that at least one blood vessel close to the surface is located under the contact surface.

Due to the high signal quality of the pulse pressure curves, even the pulse wave velocity can be determined theoretically using the dicrotic notch. For this purpose, only the geometry of the body parts examined must be measured and included in the calculation. The transit time to the reflected pulse wave is referred to as the dicrotic notch. This enables the device according to the invention to be used as a portable monitoring device for measuring biomedical data for medical purposes.

In addition, the invention relates to a method for measuring blood pressure, comprising the following steps: clamping a body part between two legs of a device for measuring blood pressure, wherein the two legs are arranged on opposite sides of a support, angled to the support, wherein a bending sensor is arranged on the support, which is designed to detect a bending of the support, and determining the blood pressure value by an evaluation unit on the basis of sensor signals of the bending sensor.

### BRIEF DESCRIPTION OF DRAWINGS:

Further advantages and details of the invention are explained below with reference to the drawings. The drawings are schematic representations and show:

FIG. 1 an embodiment of a device for measuring blood pressure according to the invention, which is attached to a patient's finger;

FIG. 2 a top view of the device shown in FIG. 1;

FIG. 3 a detail of the device shown in FIG. 1;

FIG. 4 a further detail of the device shown in FIG. 1;

FIG. 5 a further embodiment of a device according to the invention for measuring blood pressure in a tensioned state;

FIG. 6 the device shown in FIG. 5 in a relaxed state;

FIG. 7 a further embodiment of a device according to the invention;

FIG. 8 an example of a device for measuring blood pressure attached to a wristband;

FIG. 9 a further example of a device for measuring blood pressure attached to a wristband;

FIG. 10 a similar example of a device for measuring blood pressure as FIG. 9;

FIG. 11 examples of the devices shown in FIGS. 8 to 10 during blood pressure measurement;

FIG. 12 another example of a device for measuring blood pressure according to the invention;

FIG. 13 a view from below of a device for measuring blood pressure according to the invention;

FIG. 14 a sectional view of the device of FIG. 13; and

FIG. 15 a top view of the device of FIG. 13.

### DETAILED DESCRIPTION OF THE INVENTION

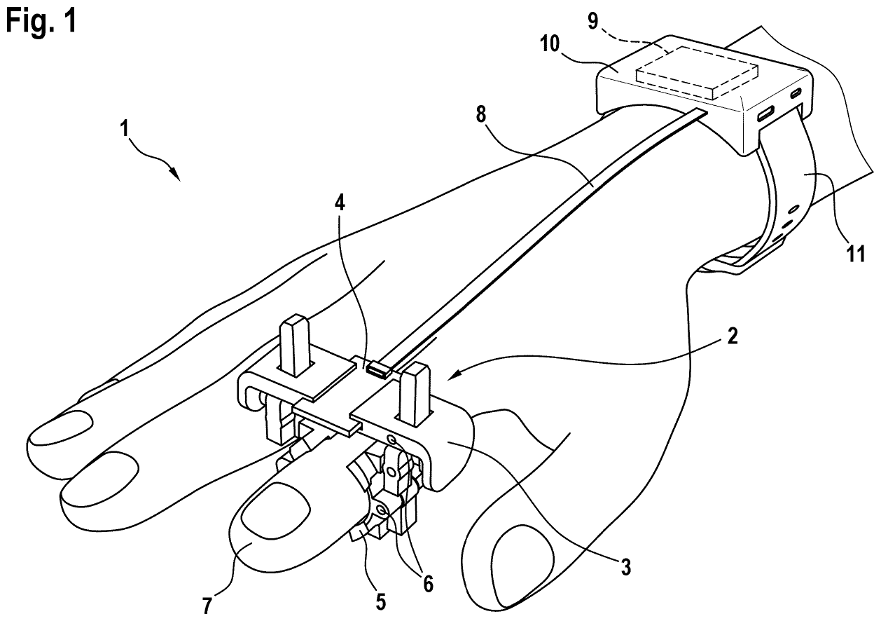

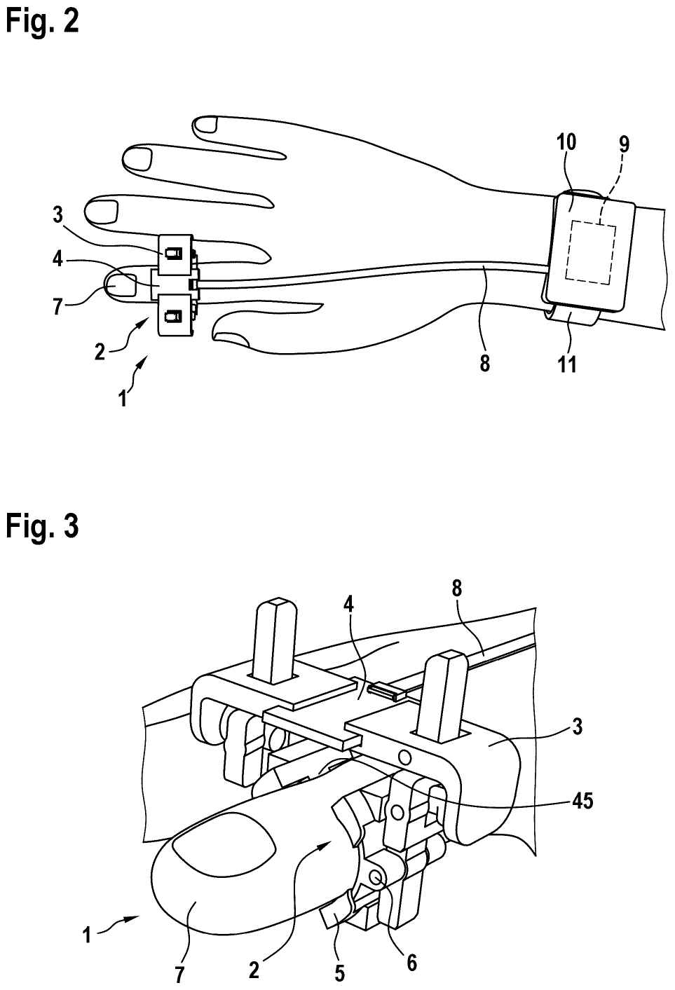

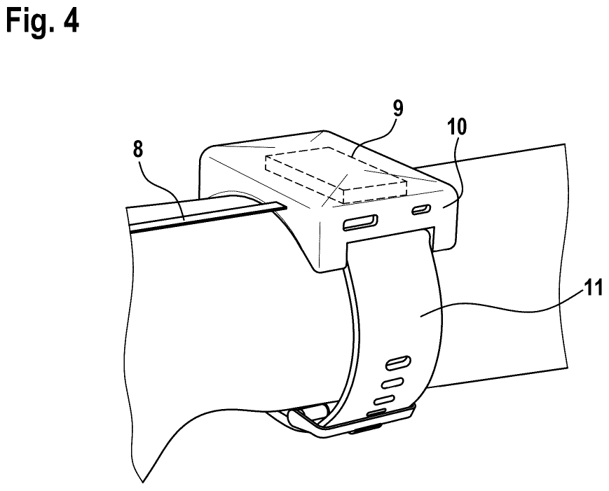

FIGS. 1 to 4 show a device for measuring blood pressure, whereby FIG. 1 shows the device attached to a patient's finger, FIG. 2 is a top view, FIG. 3 and FIG. 4 each show a detail of the device.

The device 1 comprises a two-part support 2, which has two legs 3. A bending sensor 4 is held between the two symmetrical legs 3. The support 2 formed by the legs 3 and the bending sensor 4 is U-shaped. The bending sensor 4 is a piezoelectric sensor that is designed as a bimorph sensor and has two individual sensors that are arranged around the neutral fiber of the bending sensor 4.

Each leg 3 comprises an arcuate support 5 with a narrow linear central elevation 45. The support 5 is positioned so that it is perpendicular to the patient's underlying vessels. Preferably, the support 5 is integrated into a cushion. The support 5 is pivotably mounted on the underside of the leg 3 via two joints 6. FIG. 1 shows that the shape of the curved support 5 is adapted to the outer contour of a patient's index finger 7.

The bending sensor 4 is connected to an evaluation unit 9 via a cable 8. The evaluation unit 9 comprises a housing 10 that has a wristband 11 so that the evaluation unit 9 can be worn on the wrist like a watch.

The patient's heartbeat causes arterial and venous pulsation in the blood vessels, which can also be measured on the patient's index finger. The device 1, in particular its support 5, encloses the patient's index finger 7 with a certain contact pressure or clamping force. The contact pressure or clamping force acts on the arterial or venous blood vessels running underneath in a narrow area that is defined by the shape of the support 5. The arterial and/or venous pulsation causes a minimal movement of the support 5 so that the slightly pretensioned bending sensor experiences a changing bend. The resulting bending is detected by the bending sensor 4 and converted into an electrical signal, which is transmitted to the evaluation unit 9 via the cable 8. The evaluation unit 9 uses the electrical signal to determine the blood pressure. The blood pressure value can be used in different ways. For example, it can be stored in a memory so that it is available for later evaluation. Alternatively, it can also be shown on a display. It is also possible for the evaluation unit 9 to transmit blood pressure values to another device via a wireless communication link.

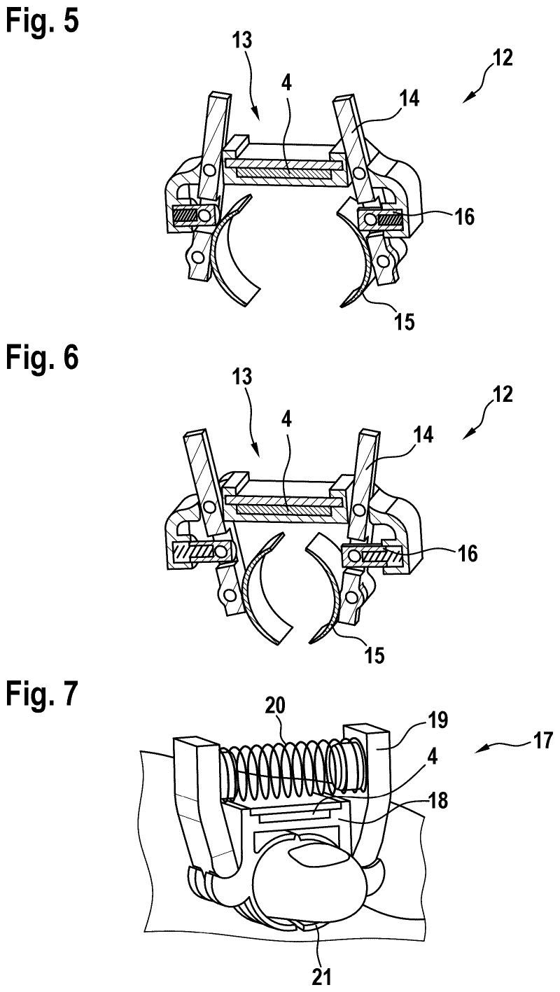

FIGS. 5 and 6 are sectional views and show a device 12 for measuring blood pressure, with FIG. 5 showing the device in a tensioned state and FIG. 6 showing the device in a relaxed state. In accordance with the first embodiment example, the device 12 comprises a U-shaped support 13 with two symmetrical legs 14, which are hingedly attached to the support 13. The bending sensor 4 is accommodated in the support 13. Each leg 14 has a curved support 15, which is shaped in such a way that both supports 15 can partially enclose a patient's finger. The two supports 15 are formed at least approximately as circular segments. The supports 15 can have a pad, which can be made of foamed plastic, rubber or a silicone material, for example. The pad has a narrow linear elevation that forms a defined point contact surface. Alternatively, a support 15 can also directly have a narrow linear elevation as a support surface. These narrow linear elevations are placed as vertically as possible on the underlying blood vessels of a patient. This results in defined couplings at the intersections between the narrow linear elevations and the blood vessels. With conventional wide pads that have no elevations, no defined coupling can occur. The pad can also contain a gel or be filled with air.

In the sectional views of FIGS. 5 and 6, it can be seen that both legs 14 each have a clamping element 16, which is designed as a spring element, more precisely as a spiral spring. The clamping element 16 is supported on the one hand on the U-shaped support 13 and on the other hand on the leg 14. When the device 12 is attached to a finger, the clamping elements 16 are compressed and exert a defined contact pressure on the finger. This state is shown in FIG. 5. On the other hand, when the device 12 is not used to measure blood pressure, the clamping elements 16 are relaxed so that the supports 15 of the legs 14 are pressed towards each other.

FIG. 7 shows another example of a device 17 for measuring blood pressure. The device 17 comprises a support 18 with the bending sensor 4 and two U-shaped legs 19, which are arranged in such a way that they are above the patient's finger during the blood pressure measurement. A spiral compression spring is arranged between the two legs 19 as a clamping element 20. At the opposite end of each leg 19 there is a support 21 for the finger. The action of the clamping element 20 pushes the legs 19 apart and the two supports 21 towards each other, so that the supports 21 are pressed against the finger with a defined force.

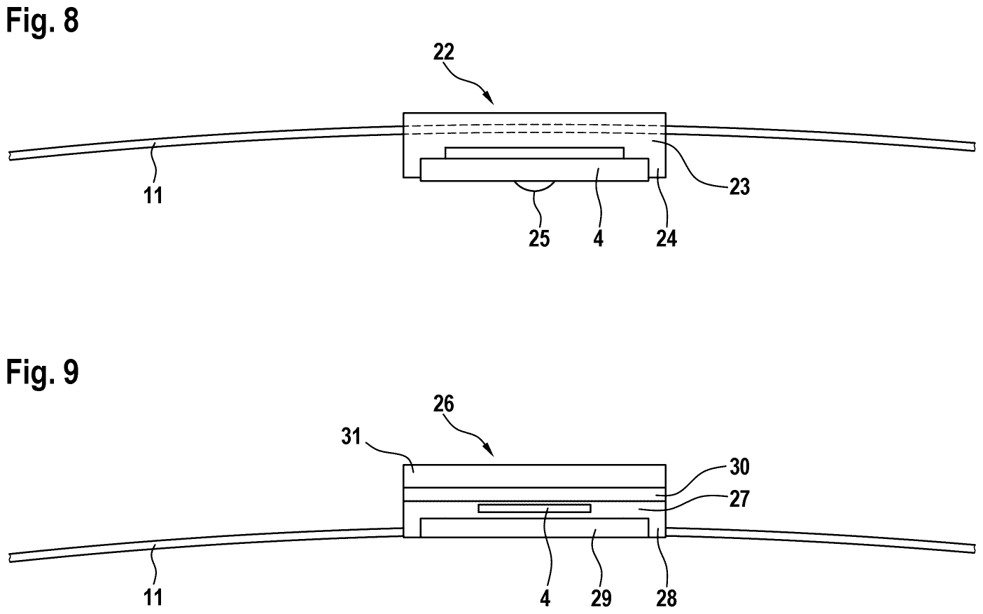

FIG. 8 shows an embodiment example of a device 22 attached to a wristband 11. The device 22 comprises a U-shaped support 23, which has the bending sensor 4. In this embodiment example, legs 24 are integrally formed on the support 23. On the side of the device 22 that can be placed on the body surface directly over a patient's artery to measure blood pressure, there is a linear support 25 on the bending sensor 4, which is shown as a circular segment or bulge in the sectioned view of FIG. 8. The support 25 is used for targeted and defined positioning of the device 22 above an artery running perpendicular to it. The device 22 can preferably be worn on the wrist.

FIG. 9 shows a similar embodiment to FIG. 8. The device 26 comprises a U-shaped support 27 with the bending sensor 4 arranged thereon. The support 27 has angled legs 28 formed integrally with it, which are attached to the wristband 11. The legs 28 enclose a pad 29 which rests on the surface of a patient's body, for example in the area of the wrist, to measure blood pressure. On the side facing outwards from the U-shaped support 27 there is a further pad 30 to which an evaluation unit 31 is attached. No support is shown on the device 26 shown in FIG. 9. In other embodiments, a support may be located at a position of the wristband 11 which is directly above an artery or vein when the wristband is put on.

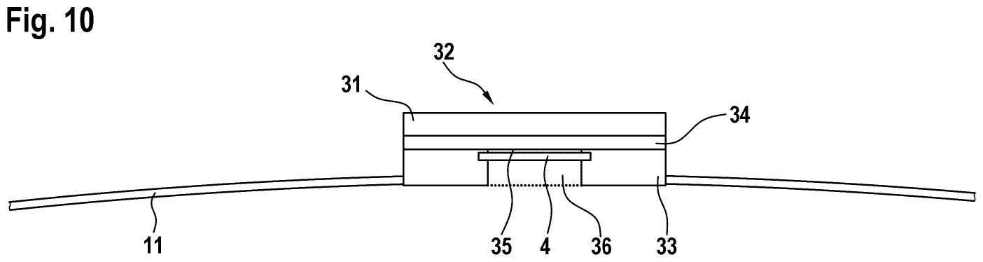

FIG. 10 shows a similar device 32 to the device 26 shown in FIG. 9. However, the legs 33 forming a support, between which the bending sensor 4 is arranged, are separate. On the outward-facing side of the legs 33 there is a pad 34 to which the evaluation unit 31 is attached. There is a pad 35 on the outside of the bending sensor 4 and a pad 36 on the inside, which fills the free space between the legs 33. In the device 26 shown in FIG. 10, no support is shown either. In other embodiments, a pad may be located at a position of the armband 11 which, when applied, is located directly over an artery or vein.

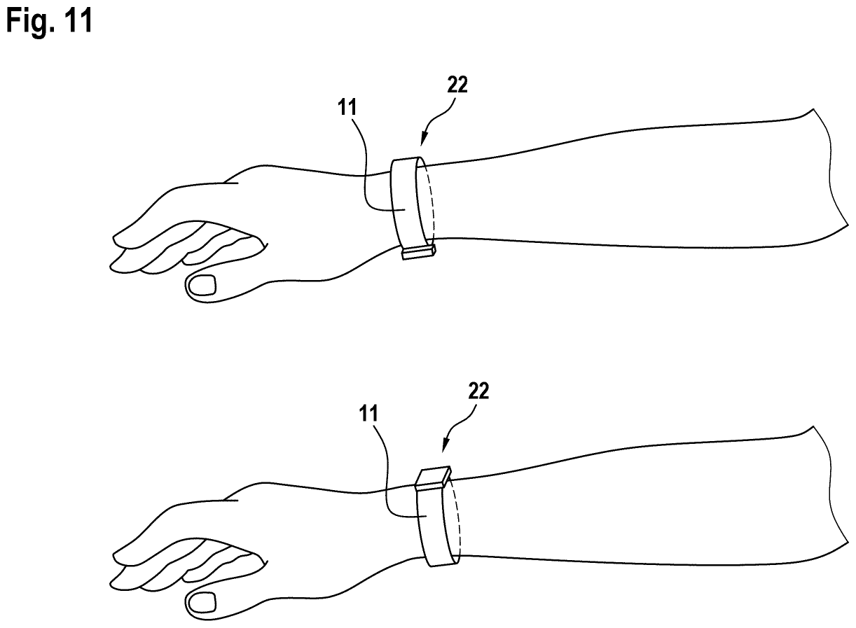

FIG. 11 shows examples of the devices shown in FIGS. 8 to 10 during blood pressure measurement. In the upper part of FIG. 11, the device 22 is shown, the wristband of which is located on the wrist of a patient. The device 22 for measuring blood pressure is located on the inside of the patient's wrist, directly above the arterial or venous blood vessels. Instead of the device 22, the previously described devices 26 and 32 can also be attached to the opposite position, i.e. to the outside of the wrist. In the lower part of FIG. 11, the device 22 is located on the outside of the wrist and is worn similarly to a wristwatch.

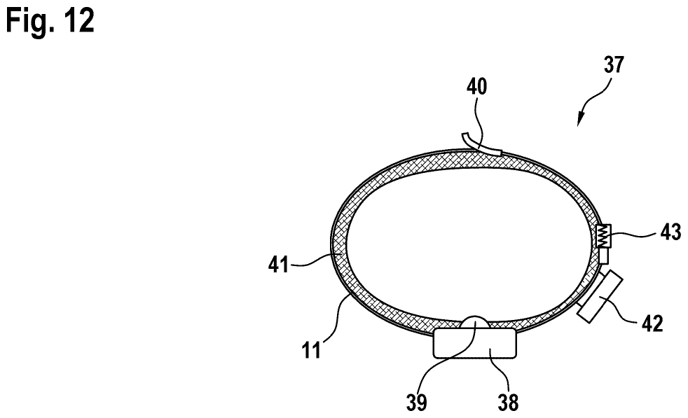

FIG. 12 shows an example of a device 37 with a wristband 11, a support 38, which is only shown schematically and has the bending sensor (not shown). On the inward-facing side, the support 38 has a support 39. The wristband 11 has a fastening element 40, for example a buckle or a Velcro fastener. On its inner side, the wristband 11 is provided with a pad 41. In addition, the wristband 11 comprises a device 42 for setting a tension, whereby the tension can be set either manually or automatically. The wristband 11 also has a display 43 for displaying the tension or the fastening force.

Several variations of the device 37 are possible. For example, the support 39 can also be arranged at a position remote from the support 38, for example opposite the support 38, in the vicinity of the fastening element 40. The pad 41 on the inside of the wristband 11 is optional and can therefore also be omitted.

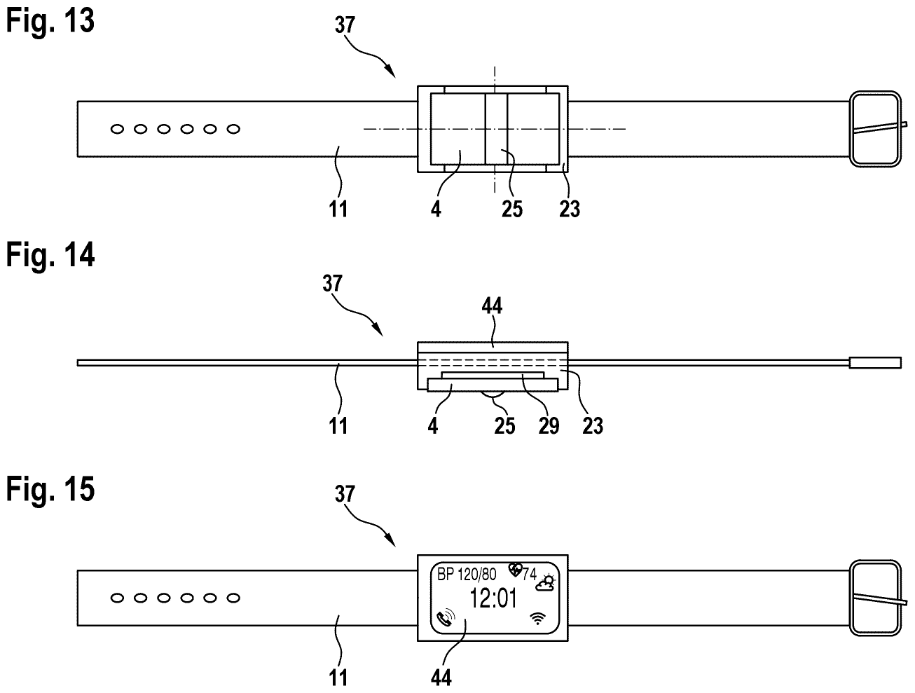

FIGS. 13 to 15 show an embodiment example of the device 37, where FIG. 13 is a view from below, FIG. 14 is a sectioned view and FIG. 15 is a top view. The device 37 comprises the wristband 11, on which the support 23 with the bending sensor 4 is located. A narrow strip-shaped support 25 is located on the underside of the bending sensor 4. A pad 29 is located in a free space between the legs of the wearer 23. The device 37 is designed as a “smartwatch”. In addition to blood pressure values, a display 44 can show, for example, the pulse and other information-similar to a smartphone.

### LIST OF REFERENCES

1 device
2 support
3 leg
4 bending sensor
5 support
6 joint
7 index finger
8 cable
9 evaluation unit
10 housing
11 wristband
12 device
13 support
14 leg
15 support
16 clamping element
17 device
18 support
19 leg
20 clamping clement
21 support
22 device
23 support
24 leg
25 support
26 device
27 support
28 leg
29 pad
30 pad
31 evaluation unit
32 device
33 leg
34 pad
35 pad
36 pad
37 device
38 support
39 support
40 fastening element
41 padding
42 device
43 display
44 display
45 elevation

1. A device (1, 12, 17) for blood pressure measurement on a part of the body with blood vessels, comprising:
a support (2, 13, 18),
a bending sensor (4) arranged on the support (2, 13, 18), which is designed to detect a bend in the support (2, 13, 18),
an evaluation unit (9) which is designed to determine a blood pressure value using sensor signals from the bending sensor (4),
characterized in that the support (2, 13, 18), in which the bending sensor (4) is accommodated, is U-shaped, and comprises two legs (3, 14, 19), which are arranged on opposite sides of the support (2, 13, 18) at an angle to the support (2, 13, 18), wherein the bending sensor (4) is held between the two legs (3, 14, 19), wherein the legs (3, 14, 19) each have a support (5, 15, 21), wherein one leg (3, 14, 19) or both legs (3, 14, 19) are arranged in an articulated manner on the support (2, 13, 18),
wherein the device (1, 12, 17) is designed such that a movement of the support (5, 15, 21), caused by an arterial and/or venous pulsation of the blood vessels of the body part, causes a bending of the bending sensor (4), via the legs (3, 14, 19) of the U-shaped support (2, 13, 18), wherein said bending is converted into the sensor signal.

2. The device according to claim 1, wherein the bending sensor (4) is a piezoelectric sensor.

3. The device according to claim 1, wherein the bending sensor (4) is designed as a bimorph sensor arrangement with two individual sensors arranged around the neutral fiber.

4. The device according to claim 1, wherein the bending sensor (4) is designed as a multimorph bending sensor and has several pairs of individual sensors with alternating antiparallel polarity.

5. (canceled)

6. The device according to claim 1, wherein the legs (3) each have a support (5, 15, 21) which is in the shape of a segment of a circle.

7. The device according to claim 1, wherein at least one clamping element (16, 20) is provided in order to apply a defined force to a leg (14, 19).

8. The device according to claim 7, wherein a clamping element (16, 20) is assigned to each leg (14, 19).

9. The device according to claim 7, wherein the clamping element (16, 20) has a spring element by means of which either one leg (14, 19) or both legs (14, 19) can be subjected to a force.

10. The device according to claim 7, wherein the force exerted by the clamping element (16, 20) is adjustable.

11. The device according to claim 7, wherein a clamping element (16, 20) is supported on one side on the support (13) and on the other side on a leg (14).

12-13. (canceled)

14. The device according to claim 1, wherein the evaluation unit (9) is designed to determine a temporal course of the blood pressure.

15. A method for blood pressure measurement, with the following steps:
clamping of a body part between two legs (3, 14, 19) of a device (1, 12, 17) for measuring blood pressure according to claim 1, the two legs (3, 14, 19) being arranged on opposite sides of a support (2, 13, 18), angled with respect to the support (2, 13, 18), wherein one leg (3, 14, 19) or both legs (3, 14, 19) are arranged in an articulated manner on the support (2, 13, 18), a bending sensor (4) being arranged on the support (2, 13, 18), which is designed to detect a bending of the support (2, 13, 18), and
determination of the blood pressure value by an evaluation unit (9) using sensor signals from the bending sensor (4).

16. The device according to claim 2, wherein the bending sensor (4) is designed as a bimorph sensor arrangement with two individual sensors arranged around the neutral fiber.

17. The device according to claim 8, wherein the clamping element (16, 20) has a spring element by means of which either one leg (14, 19) or both legs (14, 19) can be subjected to a force.

18. The device according to claim 8, wherein the force exerted by the clamping element (16, 20) is adjustable.

19. The device according to claim 9, wherein the force exerted by the clamping element (16, 20) is adjustable.

20. The device according to claim 8, wherein a clamping element (16, 20) is supported on one side on the support (13) and on the other side on a leg (14).

21. The device according to claim 9, wherein a clamping element (16, 20) is supported on one side on the support (13) and on the other side on a leg (14).
